(19) United States
(12) Patent Application Publication
(10) Pub. No.: US 2018/0110923 A1
(43) Pub. Date: Apr. 26, 2018

(54) SYSTEM AND METHODS FOR OVERDOSE MITIGATION

(71) Applicant: ARKHAM ENTERPRISES, LLC, Dover, DE (US)

(72) Inventors: Irving D. Kaplan, Dedham, MA (US); Edward J. Holupka, Medway, MA (US)

(21) Appl. No.: 15/695,618

(22) Filed: Sep. 5, 2017

Related U.S. Application Data
(60) Provisional application No. 62/411,069, filed on Oct. 21, 2016.

(51) Int. Cl. A61M 5/20; A61K 31/485

(52) U.S. Cl. CPC A61M 5/20; A61K 31/485; A61M 2205/18; A61M 2230/20; A61M 2205/581; A61M 2005/206; A61M 2205/50; A61M 2205/054; A61M 2205/8206; A61M 2209/088; A61M 2205/502

(57) ABSTRACT

Systems and methods for mitigating or preventing opioid overdoses are disclosed herein. More specifically, an overdose mitigation system includes an overdose sensor, such as a pulse oximeter, that is strapped around a user's arm in a similar manner as a blood pressure cuff. The system measures an overdose indicator, such as a minimum oxygen saturation level. When the indicator is detected, the system sounds an alarm and automatically injects an opioid antidote (e.g., intramuscularly), via an attached injector with a reservoir containing the antidote, unless an input is received from a user interface. Thus, systems and methods disclosed herein allow for an antidote to be automatically delivered to a user at risk of death from overdose, without having to wait on a first responder or rely on a caregiver.

### CROSS-REFERENCE TO RELATED APPLICATIONS

This application claims the benefit of U.S. Provisional Application Ser. No. 62/411,069, filed Oct. 21, 2016, and entitled SYSTEMS AND METHODS FOR OVERDOSE MITIGATION, the entirety of which is hereby incorporated by reference.

### STATEMENT REGARDING FEDERALLY SPONSORED RESEARCH OR DEVELOPMENT

Not Applicable

### FIELD

The present disclosure relates to systems and methods for preventing or mitigating accidents associated with overdoses. More specifically, disclosed embodiments relate to preventing death from drug overdoses, such as opioid overdoses, by automatic delivery of a narcotic antidote within seconds of an overdose.

### BACKGROUND

Despite distribution of narcotics, or opioids, being strictly controlled and, in many instances, illegal, use of narcotics still occurs. Deaths due to accidental narcotics overdoses are a major preventable cause of death. This high rate of overdose deaths occurs in spite of attempts to widely distribute an overdose antidote (e.g., Naloxone), including attempts to widely distribute the antidote to first responders and care givers. The mechanism of death in overdose is generally due to respiratory suppression, which leads to respiratory arrest, hypoxia, and death. Thus, rapid administration of an antidote is critical to prevent deaths due to opioid overdoses.

One example of an antidote is Naloxone, which is an opioid antagonist. It works by binding to opioid receptors in the brain, and can block the effects of narcotics. This leads to a rapid reversal of opiate effects in the central nervous system. Naloxone can be administered intravenously, intranasally, or intramuscularly. An adult dose of Naloxone for an opioid overdose may range from 0.4 to 2 mg/dose and may be repeated every 2 to 3 minutes as needed, up to a maximum cumulative dose (e.g., of around 10 mg). Naloxone is part of overdose kits and has been shown to reduce the number of overdose deaths. However, the overdose victim is unable to self-administer the medication.

### SUMMARY

According to some embodiments, an overdose mitigation system includes an injector coupled to a reservoir containing a narcotic antidote. The system includes an overdose sensor for detecting an overdose indicator. The system includes a processor coupled to a user interface, an alarm, and the injector. The processor is configured to, in response to detection of the overdose indicator: actuate the alarm; wait a predetermined amount of time; and actuate the injector unless an input is received from the user interface.

According to some embodiments, an overdose mitigation method includes detecting an overdose indicator with an overdose sensor. The method includes, in response to detecting the overdose indicator, actuating an alarm, waiting a predetermined amount of time, and delivering an antidote via an injector with a reservoir containing a narcotic antidote unless an input is received from a user interface device.

### BRIEF DESCRIPTION OF THE DRAWINGS

FIG. 1 is a simplified block diagram showing components of an overdose mitigation system, according to some embodiments.

FIG. 2 illustrates a flow chart of a method, according to some embodiments.

### DETAILED DESCRIPTION

The following detailed description includes references to the accompanying figures. The example embodiments described herein are not meant to be limiting. Other embodiments may be utilized, and other changes may be made, without departing from the scope of the subject matter presented herein. It will be readily understood that the aspects of the present disclosure, as generally described herein and illustrated in the figures can be arranged, substituted, combined, separated, and designed in a wide variety of different configurations, all of which are contemplated herein.

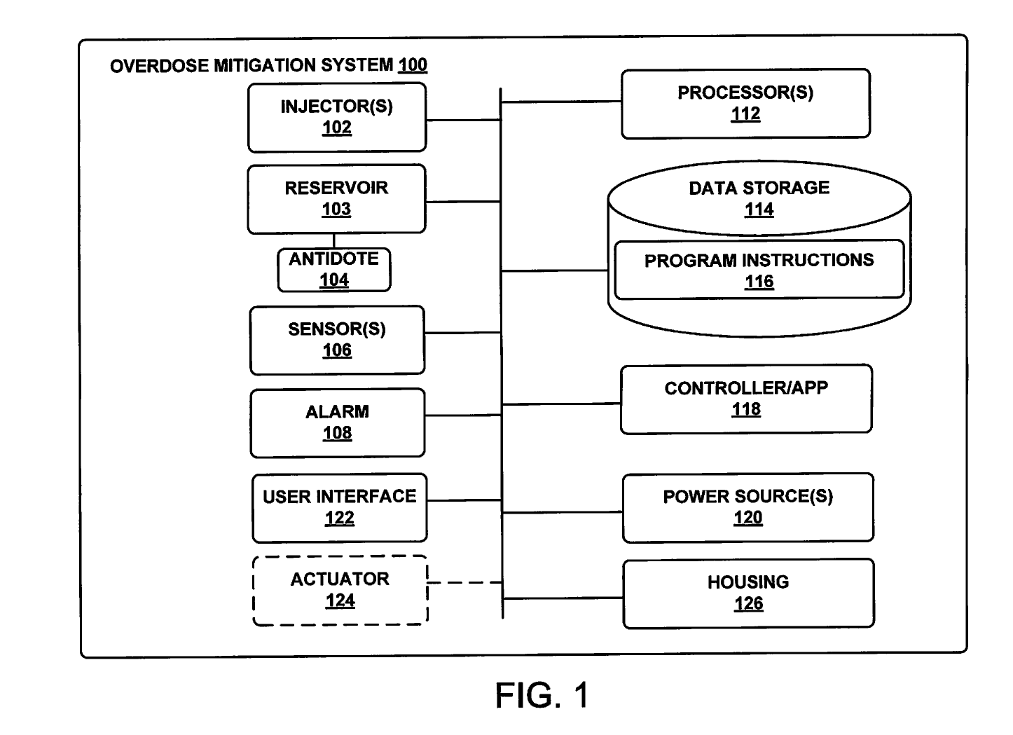

FIG. 1 illustrates a simplified block diagram showing components of an overdose mitigation system, according to some embodiments. Overdose mitigation system 100 includes injector(s) 102, a reservoir 103 for holding an antidote 104 and that is coupled with and/or in fluid communication with the injector(s) 102, sensor(s) 106, an alarm 108, processor(s) 112, data storage 114, program instructions 116, a controller/app 118, power source(s) 120, a user interface 122, actuator 124, and housing 126. The overdose mitigation system 100 is shown for illustration purposes only and may include additional components and/or have one or more components removed without departing from the scope of the disclosure. Further, the various components of overdose mitigation system 100 may be communicatively coupled or otherwise in communication with each other in any manner now known or later developed that enables the components to operate as a system to perform the functionality described herein.

Processor(s) 112 may be a general-purpose processor or a special purpose processor (e.g., digital signal processors, application specific integrated circuits, etc.). The processor(s) 112 can be configured to execute computer-readable program instructions 116 that are stored in the data storage 114 and are executable to cause the overdose mitigation system 100 to perform the functions and features described herein. For instance, the program instructions 116 may be executable to provide functionality of the controller/app 118, where the controller/app 118 may be a smartphone application that is configured to accept a touch input to turn off the alarm and stop an injection. The controller/app 118 may be configured to communicate information as well. For example, the controller/app 118 may be configured to send a text message alert or email to emergency personnel or care givers indicating an overdose has occurred (e.g., after the overdose sensor detects an overdose indicator), that the injector(s) 102 have been used, or anything else.

The data storage 114 may include or take the form of one or more computer-readable storage media that can be read or accessed by processor(s) 112. The one or more computer-readable storage media can include volatile and/or non-volatile storage components, such as optical, magnetic, organic or other memory or disc storage, which can be integrated in whole or in part with processor(s) 112. In some embodiments, the data storage 114 can be implemented using a single physical device (e.g., one optical, magnetic, organic or other memory or disc storage unit), while in other embodiments, the data storage 114 can be implemented using two or more physical devices. Further, in addition to the computer-readable program instructions 116, the data storage 114 may include additional data such as diagnostic data, among other possibilities.

The overdose mitigation system 100 may include one or more sensor(s) 106. For example, sensor(s) 106 may include a pulse oximeter sensor to measure oxygen saturation levels. The pulse oximeter may be connected to the processor(s) 112 and configured to provide an overdose indicator should oxygen saturation levels drop below a predetermined threshold. Sensor(s) 106 may be included in overdose mitigation system 100 and may provide sensor data to the processor(s) 112. For example, load sensors, position sensors, touch sensors, ultrasonic range sensors, infrared sensors, Global Positioning System (GPS) receivers, sonar, optical sensors, biosensors, force sensors, proximity sensors, Radio Frequency identification (RFID) sensors, Near Field Communication (NFC) sensors, wireless sensors, compasses, smoke sensors, light sensors, radio sensors, depth sensors (e.g., Red Green Blue plus Depth (RGB-D), lasers, structured-light, and/or a time-of-flight camera), microphones, speakers, radar, cameras (e.g., color cameras, grayscale cameras, and/or infrared cameras), and/or motion sensors (e.g., gyroscopes, accelerometers, inertial measurement units (IMU), and/or foot step or wheel odometry), among others may be used. In some embodiments, motion sensors may be used to help determine whether a user has suddenly fallen or passed out.

The overdose mitigation system 100 may include one or more alarms 108. In some embodiments, the alarm 108 is a speaker that produces a loud and audible noise after receiving an overdose indicator. The alarm 108 may also communicate (e.g., via a transmitter or the controller/app 118) with emergency personnel, care givers, or others.

Overdose mitigation system 100 may also include one or more power source(s) 120 configured to supply power to various components of the overdose mitigation system 100. Any type or combination of power source(s) 120 may be used such as, for example, one or more batteries, solar cells, or a direct, wired connection to a power source.

The user interface 122 may take various forms. In some embodiments, the user interface 122 may be a simple switch or button that the user can flip or push and indicate an input to the overdose mitigation system 100. In some embodiments, the user interface 122 may take the form of a sensor 106, such as a microphone where the user can speak and indicate an input to the overdose mitigation system 100. In some embodiments, the user interface 122 may be a graphical user interface that is integrated within the controller/app 118.

The actuator 124 may take various forms and more than one actuator 124 can be used. In some embodiments, the actuator 124 is an electroshock device that is configured to stimulate the user via shock and pain. This stimulation can be beneficial in reviving an individual from an overdose situation. In some embodiments, the actuator 124 is a speaker that plays a load noise, a transmitter that sends a help message, or a vibrating mechanism. The processor(s) 112 may actuate the actuators at periodic intervals until the overdose sensor no longer detects an overdose indicator.

In some embodiments, the overdose mitigation system 100 has a housing 126 that is an arm band device (similar to those used in a blood pressure cuff) that has an integrated wrist band pulse oximeter for its sensor 106. The arm band may be designed to part of the body (e.g., the forearm or upper arm) like a sleeve. The arm band may be held closed by Velcro straps or other means to make the arm band easy to put on and remove and facilitate the use of the system with any body type or size.

The arm band will also contain an alarm 108. If a user's oxygen saturation falls below a predetermined threshold which indicates a critical level, or an overdose indicator, the alarm 108 will produce a loud sound. If the alarm is not deactivated within a predetermined amount of time (e.g., 20 seconds, 40 seconds, or 60 seconds), a dose of an antidote such as Naxolone will be automatically delivered intramuscularly in the upper arm via a small syringe connected to the arm band device. The alarm 108 can be deactivated (thus stopping the injection) by receiving an input from the user interface 122, such as flipping a switch or pushing a button.

The injector(s) 102 may be similar to any commercially available injector or auto-injector that is designed to deliver a dose of a particular drug. In some embodiments, the injector(s) 102 may be coupled to the reservoir 103 with the antidote 104 and be placed in a non-injectable state as a default. In some embodiments, the reservoir 103 and the injector(s) 102 may be detachable from each other and from the overdose mitigation system 100 in order to be exchangeable after use or in case the antidote needs to be exchanged (e.g., if the antidote is past its expiration date and no longer approved for use).

In some embodiments, multiple injector(s) 102 may be used, and/or a single injector(s) 102 may be used that is coupled to multiple reservoirs 103, and/or the reservoir 103 may contain multiple doses of antidote, such that multiple doses of the antidote may be given. This may increase the chance of preventing death until emergency personnel or a care giver can arrive to provide further aid.

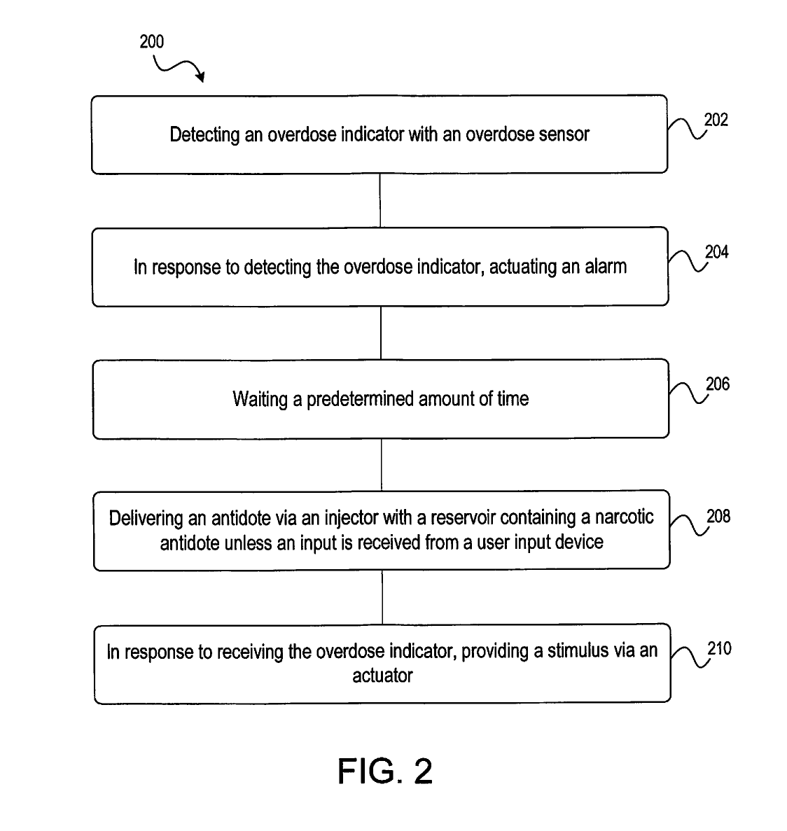

Referring now to FIG. 2, an illustrative method 200 for overdose mitigation is shown. Aspects of the method 200 may be embodied as computerized programs, routines, logic, and/or instructions executed by the overdose system 100, for example by the processor(s) 112 and one or more components of the overdose system 100, such as the injector 102. At 202, the method 200 includes detecting an overdose indicator with an overdose sensor. At 204, and in response to detecting the overdose indicator, the method 200 includes actuating an alarm. At 206, the method 200 includes waiting a predetermined amount of time which may comprise 0 seconds or 0+n seconds and may, in various embodiments, be adjusted by a user. At 208, the method 200 includes delivering an antidote via an injector with a reservoir containing a narcotic antidote, unless an input is received from a user input device. At 210, method 200 includes, in response to receiving the overdose indicator, providing a stimulus via an actuator.

While particular aspects and embodiments are disclosed herein, other aspects and embodiments will be apparent to those skilled in the art in view of the foregoing teaching. The various aspects and embodiments disclosed herein are for illustration purposes only and are not intended to be limiting, with the true scope and spirit being indicated by the following claims.

In the foregoing description, numerous specific details, examples, and scenarios are set forth in order to provide a more thorough understanding of the present disclosure. It will be appreciated, however, that embodiments of the disclosure may be practiced without such specific details. Further, such examples and scenarios are provided for illustration only, and are not intended to limit the disclosure in any way. Those of ordinary skill in the art, with the included descriptions, should be able to implement appropriate functionality without undue experimentation.

References in the specification to “an embodiment,” etc., indicate that the embodiment described may include a particular feature, structure, or characteristic. Such phrases are not necessarily referring to the same embodiment. Further, when a particular feature, structure, or characteristic is described in connection with an embodiment, it is believed to be within the knowledge of one skilled in the art to effect such feature, structure, or characteristic in connection with other embodiments whether or not explicitly indicated.

Embodiments in accordance with the disclosure may be implemented in hardware, firmware, software, or any combination thereof. Embodiments may also be implemented as instructions stored using one or more machine-readable media which may be read and executed by one or more processors. A machine-readable medium may include any suitable form of volatile or non-volatile memory.

Modules, data structures, and the like defined herein are defined as such for ease of discussion, and are not intended to imply that any specific implementation details are required. For example, any of the described modules and/or data structures may be combined or divided in sub-modules, sub-processes or other units of computer code or data as may be required by a particular design or implementation of the computing device.

In the drawings, specific arrangements or orderings of elements may be shown for ease of description. However, the specific ordering or arrangement of such elements is not meant to imply that a particular order or sequence of processing, or separation of processes, is required in all embodiments. In general, schematic elements used to represent instruction blocks or modules may be implemented using any suitable form of machine-readable instruction, and each such instruction may be implemented using any suitable programming language, library, application programming interface (API), and/or other software development tools or frameworks. Similarly, schematic elements used to represent data or information may be implemented using any suitable electronic arrangement or data structure. Further, some connections, relationships, or associations between elements may be simplified or not shown in the drawings so as not to obscure the disclosure.

This disclosure is considered to be exemplary and not restrictive. In character, and all changes and modifications that come within the spirit of the disclosure are desired to be protected.

We claim:

1. A narcotic overdose mitigation system for a person comprising:
an injector coupled with a reservoir, the reservoir containing a narcotic antidote;
an overdose sensor configured to detect an overdose indicator for the person; and
a processor coupled to a user interface, and the injector and configured to,
in response to detection of the overdose indicator,
wait a predetermined amount of time, and
actuate the injector unless an input is received from the user interface.

2. The overdose mitigation system of claim 1, further comprising an alarm, wherein the alarm is actuated in response to the overdose indicator.

3. The overdose mitigation system of claim 1, wherein the predetermined amount of time comprises zero or more seconds.

4. The overdose mitigation system of claim 1, wherein the narcotic antidote is Narcoxone.

5. The overdose mitigation system of claim 1, wherein the overdose sensor is a pulse oximeter and the overdose indicator is an oxygen saturation level.

6. The overdose mitigation system of claim 2, wherein the alarm is a speaker and actuation of the alarm comprises an audible alert.

7. The overdose mitigation system of claim 1, wherein the user interface is a switch and the input is a change in a position of the switch.

8. The overdose mitigation system of claim 2 further comprising a housing enclosing at least the injector, overdose sensor, processor, user interface, and alarm.

9. The overdose mitigation system of claim 8, wherein the housing is an arm band.

10. The overdose mitigation system of claim 1 further comprising an actuator and wherein the processor, in response to receiving the overdose indicator, is further configured to provide stimulus by actuating the actuator.

11. The overdose mitigation system of claim 10, wherein the actuator is an electroshock device.

12. The overdose mitigation system of claim 1, wherein the reservoir is detachable from the injector.

13. The overdose mitigation system of claim 1 further comprising a power source coupled to the processor, the sensor, and the injector.

14. The overdose mitigation system of claim 13, wherein the power source comprises one or more batteries.

15. An overdose mitigation method comprising:
detecting an overdose indicator with an overdose sensor; and in response to detecting the overdose indicator:
actuating an alarm;
waiting a predetermined amount of time; and
delivering an antidote via an injector with a reservoir containing a narcotic antidote unless an input is received from a user interface device.

16. The overdose mitigation method of claim 15, wherein the narcotic antidote is Narcoxone and the predetermined amount of time comprises zero or more seconds.

17. The overdose mitigation method of claim 15, wherein the overdose sensor is a pulse oximeter and the overdose indicator is an oxygen saturation level.

18. The overdose mitigation method of claim 15, wherein the alarm is a speaker and actuation of the alarm comprises an audible alert.

19. The overdose mitigation method of claim 15, wherein the user interface is a switch and the input is a change in a position of the switch.

20. The overdose mitigation method of claim 15 further comprising, in response to receiving the overdose indicator, providing stimulus via an actuator.
